(19) United States
(12) Patent Application Publication
(10) Pub. No.: US 2025/0360269 A1
(43) Pub. Date: Nov. 27, 2025

(54) METHOD AND APPARATUS FOR DETERMINING PATENCY FOR AN INTRATHECAL ACCESS SITE

(71) Applicant: EnClear Therapies, Inc., Newburyport, MA (US)

(72) Inventors: Rajan Patel, Somerville, MA (US); Anthony DePasqua, Newburyport, MA (US); Kevin Kalish, Newburyport, MA (US); Mark Guirguis, Newburyport, MA (US); Raiden Schodowski, Newburyport, MA (US); Ernesto Victor Ruiz, Allston, MA (US)

(21) Appl. No.: 19/215,901

(22) Filed: May 22, 2025

Related U.S. Application Data
(60) Provisional application No. 63/650,842, filed on May 22, 2024.

(51) Int. Cl. A61M 5/172; G16H 50/30

(52) U.S. Cl. CPC A61M 5/1723; G16H 50/30; A61M 2005/1726; A61M 2205/3334; A61M 2205/3344; A61M 2205/581; A61M 2205/582; A61M 2205/583; A61M 2205/584; A61M 2210/005; A61M 2210/0693; A61M 2230/04; A61M 2230/42

(57) ABSTRACT

Determining a patency level for an intrathecal access site includes receiving a complex cerebrospinal fluid (CSF) pressure signal for an intrathecal access site, processing the complex CSF pressure signal to extract a heart rate component and a respiratory rate component, processing the heart rate component and the respiratory rate component to determine a patency level for the intrathecal access site. An indication of the patency level may be output in any of various forms such as to automatically control a pump or other device or to provide informational input to a medical provider.

### CROSS-REFERENCE TO RELATED APPLICATION(S)

This patent application claims the benefit of U.S. Provisional Patent Application No. 63/650,842 entitled “METHOD AND APPARATUS FOR DETERMINING PATENCY” filed May 22, 2024, which is hereby incorporated herein by reference in its entirety.

The subject matter of this patent application may be related to the subject matter of one or more of the following, each of which is hereby incorporated herein by reference in its entirety:

U.S. Pat. No. 11,278,657, issued Mar. 22, 2022, and entitled “Methods of amelioration of cerebrospinal fluid and devices and systems therefor;”

United States Patent Application Publication No. US 2022/0096745 corresponding to U.S. patent application Ser. No. 17/489,633, filed Sep. 29, 2021, and entitled “Subarachnoid Fluid Conduit System and Kit;” and

United States Patent Application Publication No. US 2023/0355118 corresponding to U.S. patent application Ser. No. 18/132,174, filed Apr. 7, 2023, and entitled “SYSTEM AND METHOD FOR MONITORING PHYSIOLOGICAL PARAMETERS BASED ON CEREBROSPINAL FLUID PRESSURES TAKEN AT TWO OR MORE LOCATIONS.”

### FIELD

Illustrative embodiments of the invention generally relate to medical devices and, more particularly, various embodiments of the invention relate to managing patency of an injection site.

### BACKGROUND

The patency of an injection site generally refers to the condition of the site being open and free of obstruction, allowing for the smooth and unimpeded administration of medication or fluids. For example, it ensures that an injected needle can penetrate the skin and underlying tissues without encountering resistance, facilitating the accurate delivery of the intended substance into the body. Patency is important for the effectiveness and safety of injections, as blockages or difficulties in accessing the site can lead to improper dosing, tissue damage, or even injection-related complications.

Maintaining patency of injection sites is important for several reasons. Firstly, it ensures the proper delivery of medication or fluids, optimizing their therapeutic effects. Without a clear and accessible injection site, the intended substance may not reach its target in the body at the desired rate or concentration, potentially compromising treatment outcomes. Secondly, preserving patency minimizes the risk of injury or discomfort for the patient. A blocked or obstructed site can cause pain, bruising, or tissue damage during injection, leading to patient discomfort and potential reluctance to undergo further treatment. Overall, prioritizing patency helps to ensure the efficiency, safety, and comfort of the injection process, contributing to better patient care and treatment outcomes.

### REFERENCES TO RELEVANT PAPERS

Reference 1. Vuillemenot, et al., Safety Evaluation of CNS Administered Biologics—Study Design, Data Interpretation, and Translation to the Clinic, Toxicological Sciences, Volume 152, Issue 1, July 2016, Pages 3-9, https://doi.org/10.1093/toxsci/kfw072.
Reference 2. Ghazal Naseri Kouzehgarani. Harnessing cerebrospinal fluid circulation for drug delivery to brain tissues. Advanced Drug Delivery Reviews 173 (2021) 20-59.
Reference 3. Wagshul et al. The pulsating brain: A review of experimental and clinical studies of intracranial pulsatility, Fluids and Barriers of the CNS 2011, 8:5.
Reference 4. Sullivan et al. Convective forces increase rostral delivery of intrathecal radiotracers and antisense oligonucleotides in the cynomolgus monkey nervous system, J Transl Med (2020) 18:309.
Reference 5. Nag DS, Sahu S, Swain A, Kant S. Intracranial pressure monitoring: Gold standard and recent innovations. World J Clin Cases. 2019 Jul. 6;7 (13): 1535-1553. doi: 10.12998/wjcc.v7.i13.1535. PMID: 31367614; PMCID: PMC6658373 (https://pubmed.ncbi.nlm.nih.gov/31367614).

### SUMMARY OF VARIOUS EMBODIMENTS

In accordance with one embodiment, a method, computer program product, device, and system determines a patency level for an intrathecal access site by receiving a complex cerebrospinal fluid (CSF) pressure signal for an intrathecal access site, processing the complex CSF pressure signal to extract a heart rate component and a respiratory rate component, and processing the heart rate component and the respiratory rate component to determine a patency level for the intrathecal access site.

The patency level may be based on amplitude of at least one of the heart rate component or the respiratory rate component. The complex CSF pressure signal may be received from an intrathecal access device such as a catheter or syringe (wherein the intrathecal access device may include a needle) or may be received from an in-line fluid sensor. Processing the complex CSF pressure signal may involve passing the complex CSF pressure signal through a low-pass filter prior to processing. Processing the complex CSF pressure signal may involve an FFT analysis to produce two peaks with one peak being a low frequency peak corresponding to the respiratory rate and a second peak being a high frequency peak corresponding to the heart rate, which may involve applying a filter to the heart rate signal and the respiration rate signal (e.g., an infinite or finite response filter) or may involve counting the frequency of the filtered signals in real time after applying a filter. Processing the complex CSF pressure signal may involve a peak/valley process to derive the respiratory rate and the heart rate. Processing the complex CSF pressure signal may involve fitting and approximating a sine wave to a time domain pressure signal as a sinusoidal regression, the regression having a correlation of determination (R2) above a predetermined minimum or threshold. The patency level may include at least one of a binary characterization (e.g., patent vs. not patent), a trinary characterization (e.g., patent vs. not patent vs. inconclusive), a scalar characterization (e.g., X units on a specified scale), or a numerical characterization (e.g., X % patency). An indication of the patency level may be output, e.g., including at least one of a graphical indication (e.g., using words, color, bar graph, gauge, or other indication on a display device via a graphical user interface), a visual indication (e.g., turning on a light if patency if absent or lost, or using different color lights to indicate patency level such as green for patent, yellow for inconclusive, and red for no/inadequate patency), an audible indication (e.g., a buzzer if patency is absent/inadequate or lost, or a spoken audible indication of the patency level), or a tactile indication (e.g., a tactile alert such as vibration of a device worn by a caregiver if patency is absent or lost). A pump or other device may be controlled based on the patency level such as automatically stopping the pump when patency level falls below a predetermined threshold or automatically controlling pump flow rate based on patency level (e.g., reduce flow rate if patency level is reduced or increase flow rate if patency level increases).

Any of various types of devices (e.g., a capital device such as an infusion pump, a “smart” access device such as a catheter or syringe, a “smart” may be performed in a capital device such as an infusion pump, in a “smart” access device such as a catheter or syringe, in a “smart” in-line fluid sensor, or other types of devices) may include a sensor system for sensing and providing the complex CSF pressure signal, a processor system for performing the processing to determine the patency level, and/or an output system for outputting an indication of the patency level or for controlling a pump or other device based on the patency level. For example, a device may include one, two, or all three of such systems. Devices additionally may include any of various types of patency level indication devices (e.g., displays for a graphical user interface, one or more LEDs, etc.). Devices additionally may include a pump or other device that is controlled based on the patency level.

In accordance with another embodiment, a method and/or apparatus receives a heart rate signal and a respiration rate signal of a patient. After receipt, the method uses those two signals to determine patency at an injection site.

Some embodiments apply a Fast Fourier Transform (“FFT”) to the heart rate signal and the respiration rate signal to produce two peaks. In this case, one of those peaks is a low frequency peak and a second of those peaks is a high frequency peak. Some embodiments apply a filter to the heart rate signal and the respiration rate signal. In addition, the method may count the frequency of the filtered signals in real time after applying a filter. Among other types, the filter may be one or both of an infinite or finite response filter.

The method may fit and approximate a sine wave to a time domain pressure signal as a sinusoidal regression. For example, the regression may have a correlation of determination (R2) above a certain minimum or threshold. Moreover, the method may display an interface having the determined patency.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 graphically shows signals exemplifying physiologic waveforms of the types used in illustrative embodiments.

FIG. 2 is a flow chart for outputting an indication of patency level based on raw pressure input signals, in accordance with various embodiments using the FFT process or the peak/valley process.

FIGS. 3A and 3B demonstrate how one such study took place during bolus infusion in non-human primates (NHPs).

FIG. 4 shows waveforms and experimental data for the study shown in FIG. 3B in accordance with illustrative embodiments.

FIG. 5 graphically shows a compliance calculation in accordance with illustrative embodiments.

FIG. 6 shows a process control loop to dictate infusion rate of a therapeutic in accordance with illustrative embodiments.

FIG. 7A shows a peak/valley process of determining heart and respiration parameters in accordance with illustrative embodiments.

FIG. 7B graphically shows an example of the process of FIG. 7A.

FIG. 8 depicts an example of moving average pressure vs. time where patency is present in both the right ventricle (top graph) and left ventricle (bottom graph), as indicated by presence of both HR and RR components of sufficient amplitude.

FIG. 9 depicts an example of moving average pressure vs. time where patency is present in the right ventricle (top graph), as indicated by presence of both HR and RR components of sufficient amplitude, but not the left ventricle (bottom graph), as indicated by insufficient HR and RR component amplitudes.

FIG. 10 depicts an example of moving average pressure vs. time where patency is lost in the left ventricle (bottom graph), which could be caused, for example, by a blockage or movement of the access device.

FIG. 11 shows an example FFT process in accordance with certain embodiments.

FIG. 12A depicts a patency system using an in-line sensor device in accordance with certain embodiments.

FIG. 12B depicts a “smart” syringe device having integrated LEDs to indicate patency level in accordance with certain embodiments.

FIG. 12C depicts a “smart” syringe device that sends patency information to an external system such as the Enclear system in accordance with certain embodiments.

FIGS. 13A and 13B depict a “smart” in-line fluid sensor device in accordance with certain embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Embodiments of illustrative embodiments determine a patency level for an intrathecal access site based on heart rate and respiration rate signals derived from a complex cerebrospinal fluid (CSF) pressure signal. Illustrative embodiments use a platform for drug delivery to the central nervous system (“CNS”) through controlled flow of the cerebrospinal fluid (“CSF”) via implantable access, although alternative embodiments can be implemented in other ways such as discussed below. Experimenters have demonstrated feasibility with a prototype capital and disposable system that is capable of bi-directional flow with pre-programmable flow profiles. In some embodiments, an instrumented disposable (e.g., catheter, syringe, in-line fluid sensor, etc.) provides sensors that measure real-time pressure, flow, temperature, and air bubble data, which are acquired, stored, and processed by the capital system to determine a patency level for the intrathecal access site. In certain embodiments, the capital system or the instrumented disposable includes a graphical user interface (GUI) or other interface that provides the user with an indication of the patency level. In certain embodiments, the patency level can be used to automatically control a pump or other device such as to stop the device if patency level falls below a predetermined threshold or to control a fluid flow rate based on patency level (e.g., reduce flow rate if patency level is reduced or increase flow rate if patency level increases).

While the pressure sensing system preferably maintains safety during active flow, additional applications and designs of a sensing system have been developed due to the new uses discovered. One salient example the inventors recognized was during the injection of a bolus of a drug using a needle in large animal studies, such as non-human primates (NHPs) and sheep. These bolus injections are typically done as controls to compare to active pumping from the system (sometimes referred to herein as the “Enclear” system, e.g., the EnTrega CSF Platform from EnClear Therapies as an exemplary system of a capital system in which aspects of the invention can be embodied, although aspects can be embodied in other systems) and are analogous to the standard of care in humans. The process of locating the desired site of injection, such as the lateral ventricle in the brain, is not straightforward. As such, this process often involves the use of advanced imaging systems, such as Fluoroscopy, MRI, and real time imaging devices such as Brain-Sight. Even with these systems in place, it is still difficult to understand if the needle is correctly in the vessel, and if it is patent.

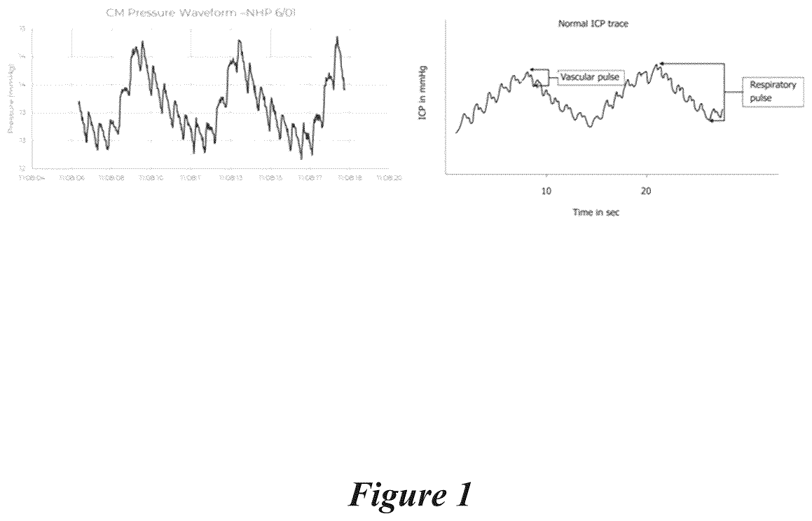

At the outset of some work, a surgical team was using priming fluid pooled in the access needle and was attempting to visually determine if the fluid in the syringe was shaking or moving in time with the heart rate. Since then, a veterinary surgical team has started using the Enclear pressure sensing system to determine if the needle is in place by using the graphical interface, which displays the pressure waveform. If the pressure waveform shows the typical shape of physiologic signals such as those in FIG. 1 showing an example of raw pressure signals (left) and an example of intracranial pressure measurement signals from Reference 5 (right), then the surgical team can be confident that the vessel has been accessed and that it is patent. Conversely, if the pressure wave form is flat, or inconclusive, the surgical team can take steps such as adjusting the needle position (such as moving the needle deeper or shallower or rotating the needle) until the waveform displays the physiologic signal. A similar process was undertaken during an injection via catheter implanted in the cisterna magna. In this scenario, a catheter was placed three days prior to the injection, and patency was assessed by the surgeon attempting to pull and push priming fluid in the implanted catheter with a syringe and feeling for resistance.

With this in mind and to reduce complexities, the inventors developed a way of determining a level of patency for an intrathecal access site (e.g., lateral ventricle, lumbar, cisterna magna, etc.) by extracting physiologic waveforms in real-time from raw pressure data (e.g., from an access device such as a catheter or syringe or from an in-line fluid sensor) and determining a patency level based on the extracted physiologic waveforms. An indication of the patency level may be output, which, for example, and without limitation, can be used by a medical practitioner to decide whether to proceed with injection or withdrawal through the access site or alternatively to reposition the access device for improved patency, or can be used for automated control of a pump such as to control infusion, withdrawal, or circulation rate via the intrathecal access site. For example, for the Enclear system to work effectively, access devices such as catheters or syringes in the intrathecal access points need to have good patency so that fluids such as drugs or CSF fluids can flow into or out of the access points without issues. Poor patency can result in pressure spikes and reduced or blocked flow.

In illustrative embodiments, a device, method, or system solution such as for an instrumented syringe or catheter system utilizes mathematical analysis of pressure waveforms from the intrathecal access site to characterize patency of the access site such as to inform the user that the access site is patent and is adequately accessed for best infusion. CSF moves through the CNS in a pulsatile fashion induced by the heart rate and respiratory rate. The heart rate (HR) and respiratory rate (RR) are also present in the pressure waveform in the form of a complex waveform.

In certain embodiments, this complex waveform is processed to extract the HR and RR components and then the HR and RR components are used to characterize a level of patency of the access site, which in some embodiments can be done on an ongoing basis (e.g., making an initial characterization upon insertion of the access device and then making additional characterizations over time such as during injection or withdrawal from the access site). For example, in certain embodiments, such processing includes confirming that both HR and RR components are present and determining the patency level based on amplitude of at least one of the HR component or the RR component or the entire pressure waveform, where, for example, the patency level can be determined based on the amplitude(s) falling within various predetermined thresholds or ranges where the thresholds or ranges can be absolute (e.g., the same for all patients) or relative (e.g., creating a custom threshold or range for each individual patient based on, for example, a known correlation between mean intracranial pressure and the pulsatile components of intracranial pressure or a comparison of the extracted signal to an external reference signal such as from an external heart or respiration monitor). The patency level can be characterized at any level of granularity, e.g., a binary characterization (patent vs. not patent), a trinary characterization (e.g., patent vs. not patent vs. inconclusive), a scalar characterization (e.g., X units on a specified scale), a numerical characterization (e.g., X % patency), etc. Embodiments may output an indication of the level of patency such as a graphical indication (e.g., using words, color, bar graph, gauge, or other indication on a display device via a graphical user interface), a visual indication (e.g., turning on a light if patency if absent or lost, or using different color lights to indicate patency level such as green for patent, yellow for inconclusive, and red for no/inadequate patency), an audible indication (e.g., a buzzer if patency is absent/inadequate or lost, or a spoken audible indication of the patency level), a tactile indication (e.g., a tactile alert such as vibration of a device worn by a caregiver if patency is absent or lost), or other indication. Embodiments also may output information about the complex waveform (e.g., a graphical waveform display), the HR, the RR, and other measurements and determinations.

More specifically, a CNS pressure waveform is a complex signal. Two primary components of this signal are a relatively higher frequency signal corresponding to heart rate (HR) and a relatively lower frequency corresponding to respiratory rate (RR). Because HR and RR are dynamic, patient dependent, and vary in magnitude and frequency, extracting these parameters can be challenging.

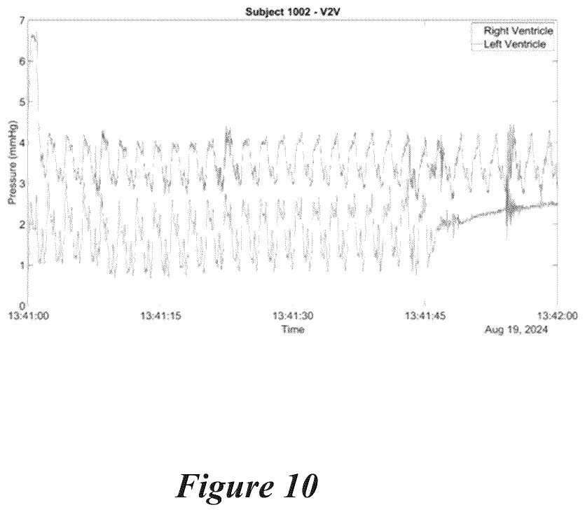

One technique for extracting the HR and RR components from the complex pressure waveform involves applying a real-time Fast Fourier Transform (FFT) to the pressure waveform to identify prominent peaks that respectively indicate the heart rate and respiratory rate. In one embodiment, this method begins with smoothing the raw waveform data via a lowpass filter with a cut-off of 3.5 Hz. Once smoothed, a Fourier transform is used to reveal the prominent waveforms. In this, the higher frequency corresponds with the HR while the lower frequency signal corresponds with the RR. With the frequency signals identified, the raw waveform is then passed through two filters which isolate them. Once isolated, the peak-to-peak values and frequencies of the HR and RR can be determined. An example of this process can be seen in FIG. 11. Utilizing the frequency, phase, and amplitude of each peak on the FFT, a sine wave can be fitted and approximated to the time domain pressure signal as a sinusoidal regression. This regression must have a correlation of determination (R2) above a certain minimum or threshold, such as 0.9, for the analysis to determine patency. Combining R2 value with other calculations, such as signal-to-noise ratio, can also help determine the reliability of the estimation. In one instance, the system can corroborate the extrapolated heart and respiratory rates with actual measured heart and respiratory rates (e.g., from an outside non-invasive monitor such as a fitness watch, pressure cuff, or pulse oximeter) and, if they are acceptably close (e.g. within 5%), then patency could be concluded (i.e., the system considers such a result to be patent) with further confidence.

This method utilizing FFT, although occurring in real-time, has an inherent lag to it because it needs a certain minimum number of periodical cycles to pass before analysis can begin. For heart rate, this lag could be a few seconds, but for respiratory rate, this lag could last upwards of 30 seconds. To extrapolate heart and respiratory rate with a quicker response time, a filter (e.g., an infinite or finite response filter) can be applied to the raw pressure waveform. This embodiment can employ two filters, namely, a low pass filter and a high pass filter respectfully corresponding to respiratory rate and heart rate. After filtering the pressure data, a frequency counter type algorithm will then count the frequency of these signals in real time and report back results. Alternatively, a feature detection algorithm, such as those employed by ECG monitors, could also calculate the frequency in real time.

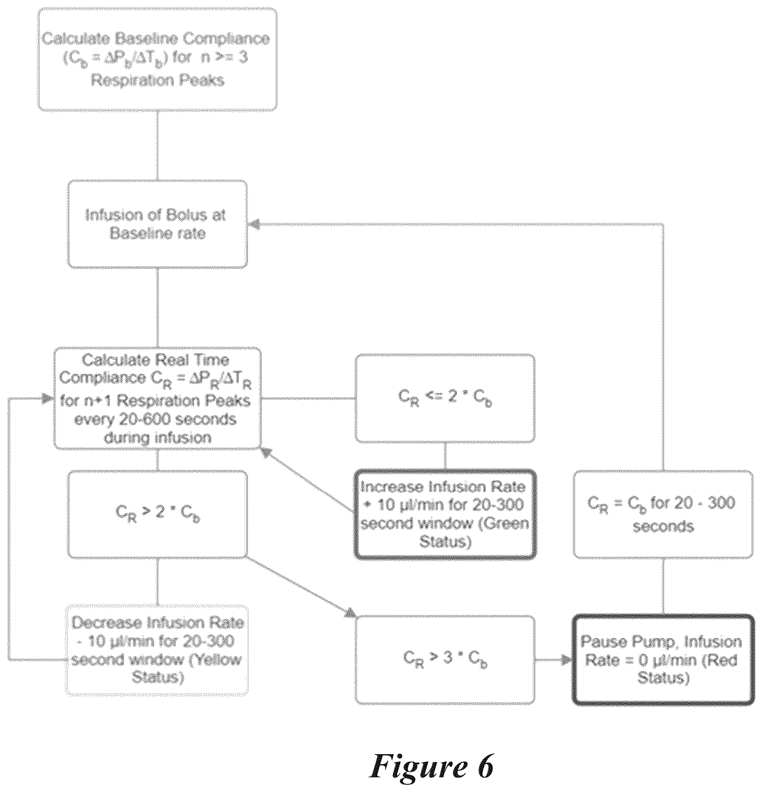

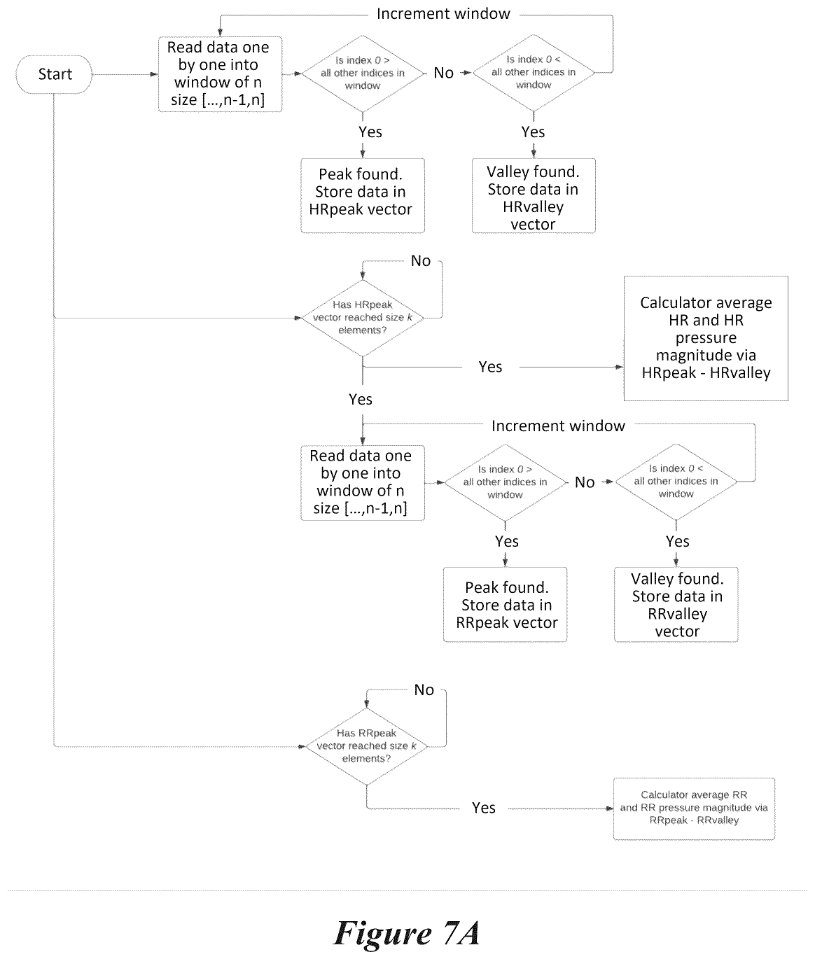

Another technique for extracting the HR and RR components from the complex pressure waveform uses a type of nested sliding window algorithm that offers two distinct advantages: real-time calculations and adaptability to magnitude and frequency fluctuations. The goal of the algorithm, which is illustrated in FIGS. 7A and 7B and is referred to herein as a peak/valley process (or alternatively as the Gurguis method, named after a primary inventor of the process), is to find the peaks of the HR and RR waveforms and then calculate their frequencies. In this embodiment, rather than converting pressure readings into frequencies such as through Fourier transforms, the pressure readings are processed to identify local peaks and valleys to determine both heart rate and magnitude and respiration rate and magnitude. Specifically, pressure readings are examined within a sequence of windows with each window containing a predetermined number of pressure readings, e.g., on a sliding window basis or by examining discrete windows. If a particular pressure reading is the greatest within a window, then that pressure reading represents a local peak. If a particular pressure reading is the lowest within a window, then that pressure reading represents a local valley. The heart or respiration rate can be determined based on the distance in time between peaks. The heart or respiration magnitude can be determined based on the differential between peaks and valleys. An exemplary embodiment of the peak/valley process involves the following steps:
i) The first step in the algorithm of one embodiment defines a set window size, n, where index 0 is the first element and index n is the last element in the window (see calculation flow in FIGS. 7A and 7B). This window follows along the real-time pressure waveform, sample for sample. Each time a sample is added to the window, the algorithm first determines if all n elements are ascending or descending. If all elements are ascending, then the window increments as it has not yet found a peak. If the window begins ascending, but then has a descending pattern, then the window advances until the 0th element is the maximum value of the window. Advancing the window until the maximum value is at the 0th element ensures that a true peak is detected and not noise in the waveform. Once the peak is found, this value is identified as a HR peak and stored in a second HR vector array. The HR vector will continue to grow as the window advances until the vector reaches k elements. Once k elements are found, an average HR is calculated based on the timestamps of each element in the HR vector. The technique repeats in real-time continuously calculating the HR frequency.
ii) The second step stores each element of the HR vector into a separate, larger array labeled the RR waveform. An identical algorithm as identified in the first step can then be processed over the RR waveform, instead of the pressure waveform, and each peak identified would be stored in a RR vector array until it reaches k elements. Once k elements are found, an average RR is calculated based on the timestamps of each element in the RR vector. The technique repeats in real-time, in conjunction with the first step, continuously calculating the RR frequency.
iii) It is important to note that the size, k, of the HR and RR vectors will dictate response time and accuracy. A smaller k means a fast response time, but potentially low accuracy in the event of the algorithm missing a peak or detecting a false peak. A larger k means a higher accuracy, but a longer response time since it needs to wait until it has collected enough datapoints.
iv) For each first and second step outlined above, the identical algorithm could be run inversely by checking that elements are descending. The resultant vectors would be valley instead of peak vectors. Then, the resulting HR and RR valley vectors could be subtracted from the corresponding peak vectors to calculate each respective waveform amplitude.
v) Determining whether a signal is valid could be done in a plurality of ways. First, if actual patient HR and RR is available (e.g., via EKG or other mechanisms), then these data could be used to corroborate the calculated data and confirm needle patency if the value matches within a certain error. Alternatively, if actual HR and RR is not available, then looking at standard deviation within HR and RR calculations could help determine if the signal is noisy.

Thus, the process shown in FIG. 7A essentially has two loops, a top loop for characterizing heart function and a bottom loop for characterizing respiration function. The input to the top loop is the “HRpeak” data vector, and the output of the top loop corresponds to the top purple curve shown in FIG. 7B, where the peaks marked with red asterisks represent the heartbeats, the distance between the peaks represent the heartbeat rate, and the differential between the peaks and valleys of that curve represent the heartbeat magnitude. The input to the bottom loop is the “RRpeak” data vector resulting from the top loop, and the output of the bottom loop corresponds to the bottom red curve shown in FIG. 7B, wherein the valleys marked with red asterisks represent the respirations, the distance between the valleys represents the respiration rate, and the differential between the peaks and valleys of that curve represents the respiration magnitude. Thus, even though the top and bottom loops use essentially the same logic, they operate on different data vectors and therefore produce different outputs, one relating to heart function and the other relating to respiration function.

In one specific embodiment, following raw waveform data obtained for total pressure, the raw data is passed through a lowpass filter with a cut-off of 3.5 Hz to smooth it and allow for easier post-process analysis. Once smoothed, the heart rate (HR) is calculated first using peak-to-peak analysis. A sliding window compares data points to each other and determines local peaks. The time interval between each of these peaks is then used to calculate the current heart rate. There can be instances of missed beats or extra beats being erroneously calculated. When this occurs, they are ignored, and only “good” peaks are used to calculate the HR. A missed or extra beat is determined by comparing its time interval to the previously established beats. If it is greater or less than the previous beats by more than 10%, it is considered a “bad” peak. The respiratory rate (RR) is found using a similar methodology. Instead of using the raw pressure data as for HR, only the peaks previously found for the heart rate are compared to each other to find the max peak within these peaks. Essentially, a second peak analysis is conducted on only the previously constructed HR peak waveform. This max peak denotes a breath, and the time interval between these is used to calculate the RR. Due to several factors like degree of patency and ICP amplitude, there are subjects whose valleys are much more defined than their peaks when looking at their pressure waveforms. When this is the case, the same process of calculating HR and RR is used but the time interval between valleys is compared as opposed to their peaks.

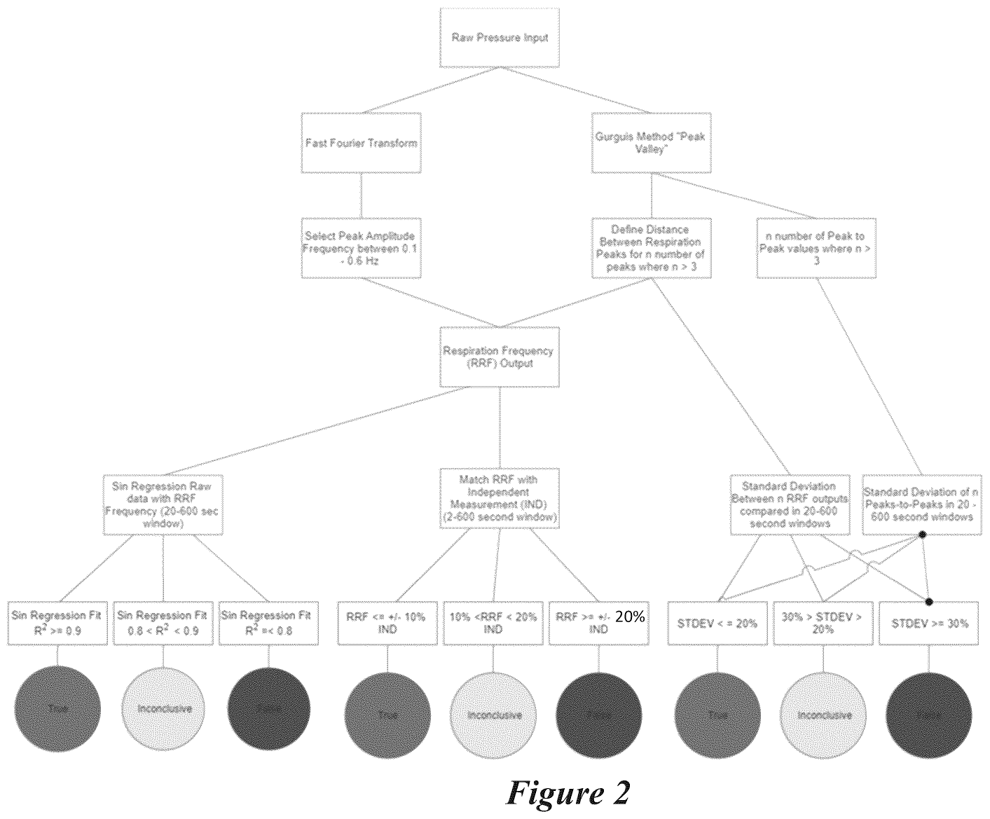

FIG. 2 is a flow chart for outputting an indication of patency level based on raw pressure input signals, in accordance with various embodiments using the FFT process or the peak/valley process. In this example, one way of using the FFT process involves fitting and approximating a sine wave to the time domain pressure signal as a sinusoidal regression and then determining a patency level based on a correlation of determination (R2) of this regression being above a certain minimum or threshold or falling within a certain range (e.g., in this example, R2>=0.9 indicates green status, 0.8<R2<0.9 indicates yellow status, and R2<=0.8 indicates red status. One way of using the peak/valley process involves comparing the RR frequency with an independent RR frequency measurement (e.g., in this example, RRF<=10% deviation indicates green status, RRF between 10% and 20% deviation indicates yellow status, and RRF greater than or equal to 20% deviation indicates red status). Another way of using the peak/valley process involves the standard deviation between n RRF outputs compared in 20-600 second windows (e.g., in this example, standard deviation less than or equal to 20% indicates green status, standard deviation between 20% and 30% indicates yellow status, and standard deviation greater than or equal to 30% indicates red status). Another way of using the peak/valley process involves the standard deviation of n peaks-to-peaks in 20-600 second windows (e.g., in this example, standard deviation less than or equal to 20% indicates green status, standard deviation between 20% and 30% indicates yellow status, and standard deviation greater than or equal to 30% indicates red status). In FIG. 2, “Green” or “True” would indicate that the access site is considered to be patent, with an access device such as a catheter or needle in CSF communicating fluid pocket, and that the user can initiate delivering their therapeutic; “Yellow” or “Inconclusive” would indicate that patency is inconclusive and there is some risk of loss of therapeutic due to sub-par placement, and it is left to the discretion of the user; and “Red” or “False” would indicate non-patency and that the user should not dose the therapeutic as the implanted catheter or needle may not be in correctly accessing a fluid pocket required for accurate dosing. It should be noted that a logic flow of the type shown in FIG. 2 could use HR instead of RR or could be based on a combination of the HR and RR or on the amplitude of the entire pressure waveform. It should be noted that the logic flow shown in FIG. 2 could be used as part of a control function such as for an infusion pump by repeating the process (e.g., green causing pumping or pumping rate to continue or increase, yellow causes pumping or pumping rate to decrease, red causes pumping to stop), in lieu of, or in addition to, providing an indication of patency level. It should be noted that a logic flow of the type shown in FIG. 2 could be modified to provide a binary indication of patency level (e.g., the path leading to green could indicate patency, while the paths leading to yellow and red could be combined to indicate non-patency).

Further, real time analysis of the waveform as the volume of therapeutic is delivered allows the user to assess the safety of the injection. This can be especially pertinent for therapies with large injection volumes such as many gene editing technologies.

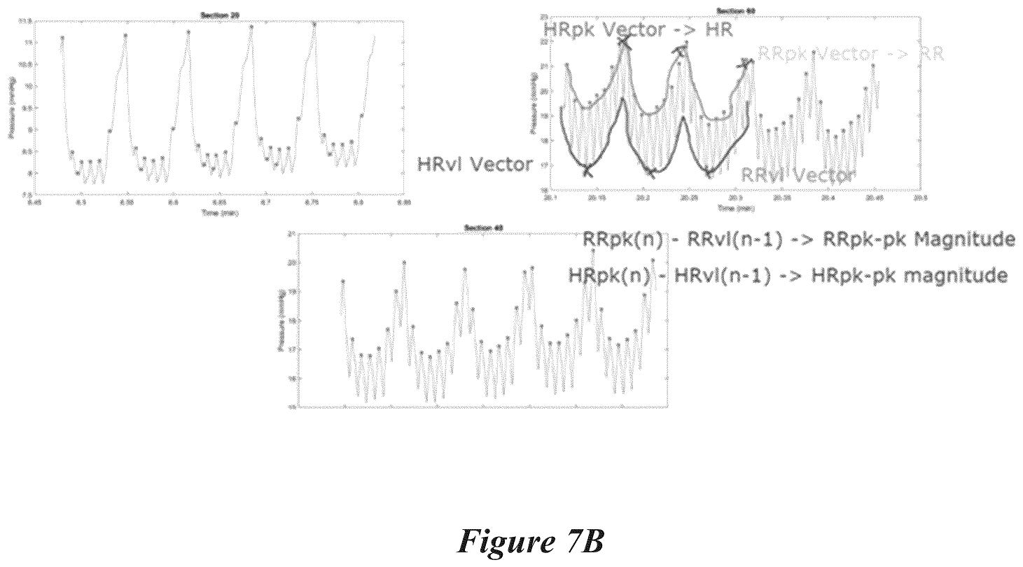

FIG. 8 depicts an example of moving average pressure vs. time where patency is present in both the right ventricle (top graph) and left ventricle (bottom graph), as indicated by presence of both HR and RR components of sufficient amplitude.

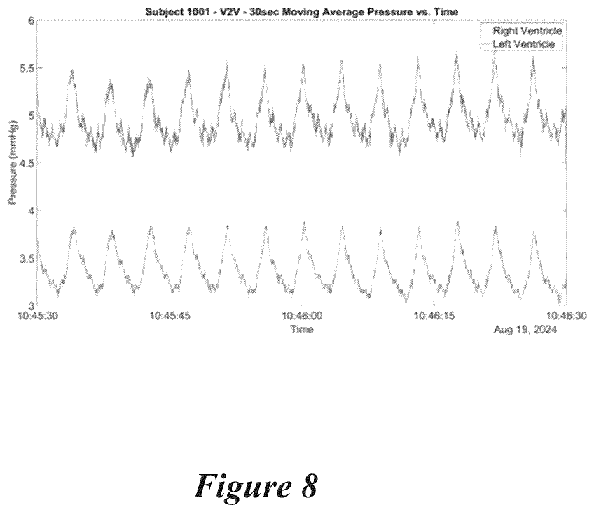

FIG. 9 depicts an example of moving average pressure vs. time where patency is present in the right ventricle (top graph), as indicated by presence of both HR and RR components of sufficient amplitude, but not the left ventricle (bottom graph), as indicated by insufficient HR and RR component amplitudes.

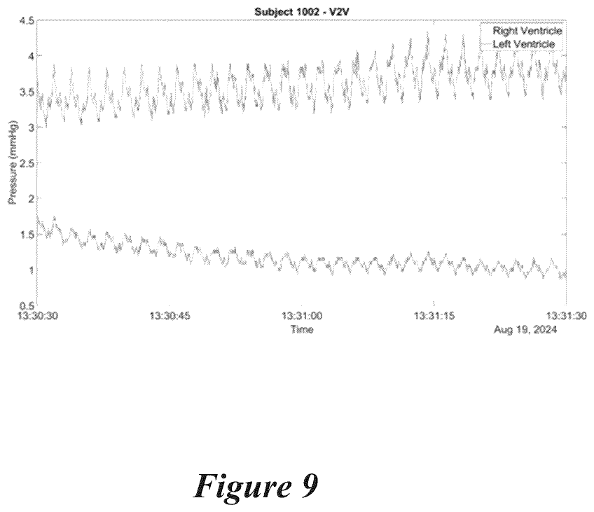

FIG. 10 depicts an example of moving average pressure vs. time where patency is lost in the left ventricle (bottom graph), which could be caused, for example, by a blockage or movement of the access device.

Thus, some embodiments use extracted physiologic signals 1) to calculate compliance of patient CNS before, during, and after infusion, and 2) as a controller for infusion rate. Specifically, next generation gene therapies are currently undergoing heavy investment and are the focus of many pharmaceutical and biotech companies and their research and development arms. Pilot experiments conducted an active arm and a control arm in which doses of a preferred animal model, typically Cynomolgus monkeys, were made, with effective delivery methodologies at that time. These control studies included the Enclear sensing system alone to characterize how CSF dynamics are affected by the current administration modality.

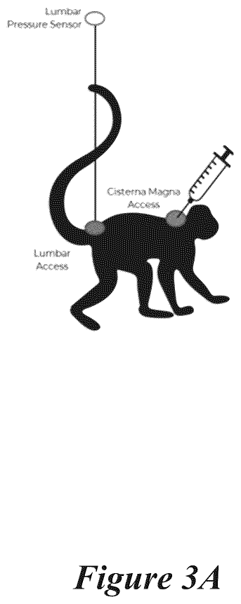

FIGS. 3A and 3B demonstrate how one study took place during bolus infusion in non-human primates (NHPs). As depicted in FIG. 3A, both catheters were inserted from the distal lumbar site. An infusion pump and drug reservoir administer therapeutic to the tip of the CM catheter, while the lumbar catheter was attached to the Enclear system. Raw lumbar pressure readings indicated a dramatic increase in CNS pressures as the bolus was delivered. However, on top of calculating and reporting nominal calculated pressures, some embodiments will report real time vessel compliance, as a therapeutic is delivered. FIG. 3B indicates raw data from a passive pressure sensor as a bolus therapeutic was delivered to the CNS, in partnered studies. Bolus dose rates, and dose volumes, were dictated by the study sponsor.

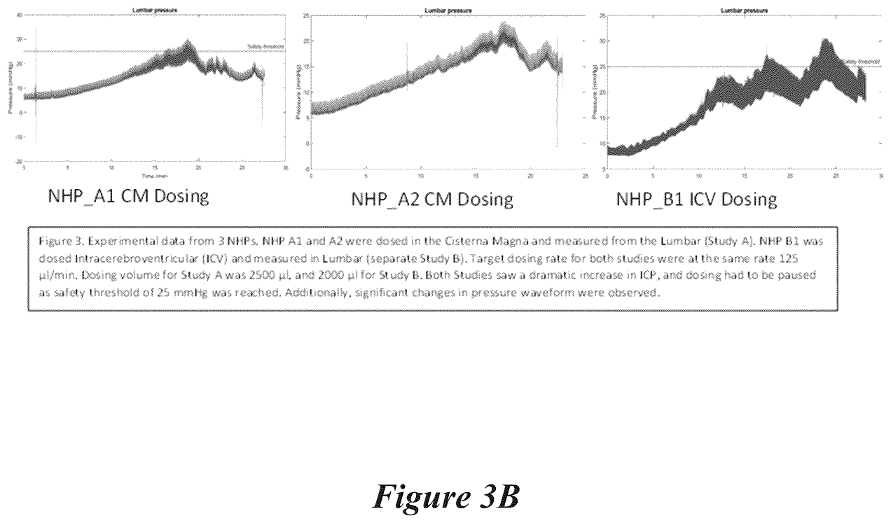

FIG. 4 depicts a waveform analysis in post process before dosing in Section 1, and when dosing had to be paused due to reaching the safety threshold in Section 2. More specifically, FIG. 4 indicates the difference in waveform analysis in 20 second windows at steady state, Section 1, before bolusing, and at maximum pressure threshold, Section 2. Note that respiratory peak to peak value and the heart rate peak to peak value for NHP A1 both include noticeable increases in conjunction with increase in nominal CNS pressure. If a prescribed protocol was followed without safety measurement and thresholds in place, it can be assumed that the pressures would have continued to increase and the waveforms to continue to increase in amplitude. This increase in amplitude can be used to measure the compliance of the CNS system. Compliance can be calculated in two ways by the Enclear system:
1) Infuse a known amount of volume of inert fluid such as artificial CSF, Saline, LSR etc. (e.g., 20-500 μl) and generate a compliance curve and a single compliance value in terms of pressure increase divided by volume. Where C=ΔP/ΔV.
2) Use the waveform analysis to compute the slope of one or more respiratory pulses or vascular pulses. Where C=ΔP/ΔT.

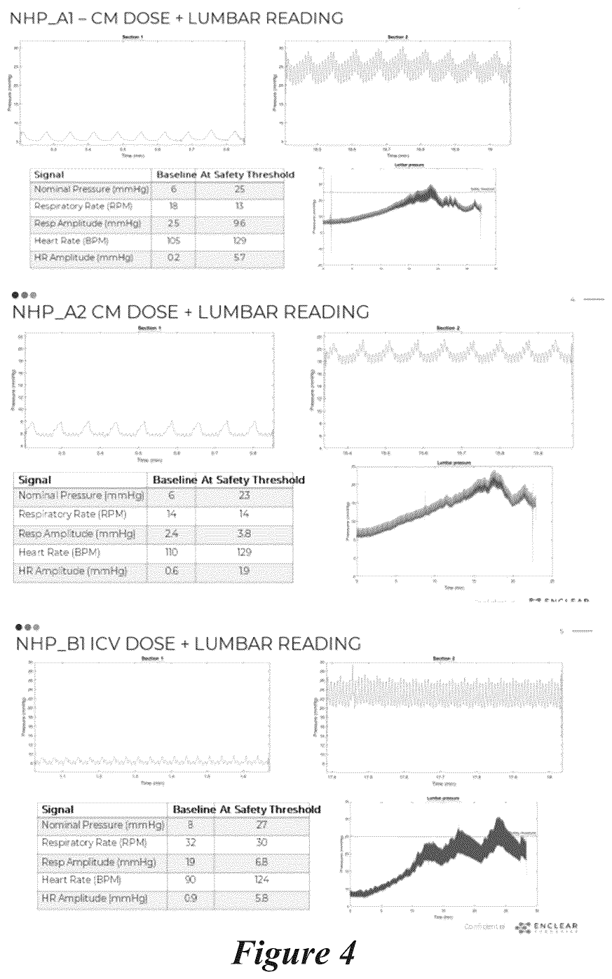

FIG. 5 indicates the compliance signal calculated from respiratory pulse for two 20 second sections of the NHP A1 graph, specifically Section 1 compliance for one peak, and Section 2 compliance for one peak. This can be a moving window in which some number (e.g., 2-3) compliance calculations on identical peaks are run and compared to a previous number (e.g., 2-3) of calculations. These compliance ratios (essentially averages) can then be used as inputs such as to generate an indication or to speed up or slow down an infusion.

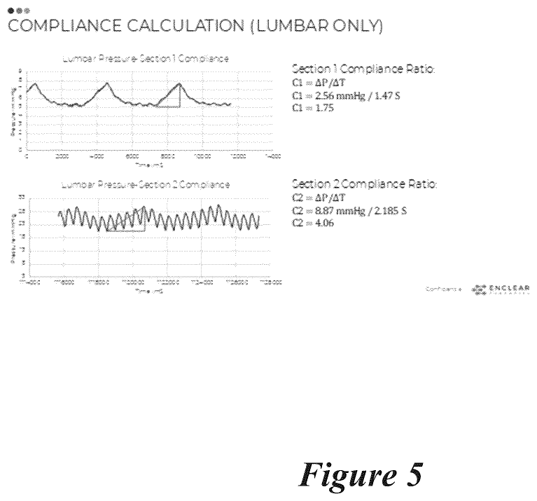

These compliance outputs of the patient's specific CNS system can be used as an input for the Enclear infusion pump system to both maintain safety and generate efficacy. For example, the compliance of the CNS system can be computed as the bolus is being infused. That compliance calculation can be used in a feedback loop to control the infusion rate and/or to indicate warnings for the user such as a red/green/yellow status indicator as described by the control loop drawn in FIG. 6. The same calculations can be used for the calculated Heart Rhythm Peaks, or by an alternate calculation by which the system calculates volume administered versus pressure increase where C=ΔP/ΔV.

The same ratios between a baseline value and a real time value can be applied to a control loop input such as for pump infusion rate, and for GUI warning signals. This can be applied such that delivery is optimized patient by patient, such that the maximum flow rate that the patient's CNS system can tolerate is achieved. This is exemplified by the difference in data recordings between NHP A1 and NHP A2 (FIG. 3B). Both NHPs were administered the same volume of drug at the same target rate. However, NHP A1 reached the safety threshold faster than the NHP A2, which never hit the threshold. In this case, NHP A2, due to CNS vessel geometry or other factors, may have been able to tolerate a faster infusion rate.

By this method, multiple control loops can be employed for precision delivery of a therapeutic to the CNS. One control loop can dictate the desired flow rate, e.g., as described by FIG. 6. The desired flow rate can then be maintained by a second control loop, such as a PID (proportional-integral-derivative) controller that uses the real time flow rate as dictated by an inline sensor as feedback to dictate voltage or RPM control of the pump motor. It should be noted that a logic flow of the type shown in FIG. 6 could use HR instead of RR or could be based on a combination of the HR and RR or on the amplitude of the entire pressure waveform. It should be noted that a logic flow of the type shown in FIG. 6 could be used to provide an indication of patency level (e.g., red/green/yellow or non-patent/patent/inconclusive) in addition to, or in lieu of, controlling a pump. It should be noted that a logic flow of the type shown in FIG. 6 could be modified to provide a binary indication of patency level (e.g., the path leading to green could indicate patency, while the paths leading to yellow and red could be combined to indicate non-patency).

In illustrative embodiments, catheter and needle systems are mechanically designed for use with these sensors and signal processors. These typically involve one or more fluid lines that are kept fluidically isolated starting from the access vessel (i.e.,—LIT or ICV CSF space), all the way to the sensor or reservoir external to the patient. The transition from internal to patient to external to patient typically occurs through a subcutaneous port, which is accessed transdermally via a specifically designed Huber needle. These needles can then terminate in standard luer connectors which can be connected to the disposable Enclear Sensing system, either in-line or parallel to the access needle. Depending on lumen configuration, one line can terminate at a drug reservoir, and the other can terminate at a dead-end pressure sensor.

In other designs, there is one lumen, and both sensors are in-line, with the pressure sensor more proximal and flow, temperature, bubble sensors more distal. This line can then still terminate at a fluidic reservoir filled with therapeutic. The reservoir can then be controlled by a capital/re-usable piece of equipment such as a lead-screw based syringe pump, which is then electronically in communication with the disposable sensors. In other embodiments, there can be as many as three lumens. In that three-lumen example, one lumen may terminate at a fluidic reservoir, another may terminate at a dead-end sensor, and a third lumen may be used for CSF recirculation, or CSF shunting. Some versions of this device use these single lumen or multiple lumen lines along with specific sensor arrays to allow for user-feedback on patency, or direct control of flow rate via digital loop.

Illustrative embodiments, including those using Enclear system designs, are configured to work in conjunction with the sensing system specifically. Fluid lines and subsequent sensor integrations can be used to better administer drugs by using the patency checking system, the pump control system, and user interface system as described in the steps above.

Further embodiments can be in the form of a manual syringe. This manual syringe can use many different versions of the sensor processing to guide the user in successful injection. In one embodiment, a disposable syringe with a simple pressure sensor fluidically connects to a three-way valve. In this use case, a user may turn the valve such that the sensor is fluidically communicating with the desired access vessel, but not fluidically communicating with the drug reservoir. After the signal sensing system has said that the vessel has been successfully accessed (e.g., via green light), the user can then turn the valve such that the sensing system is no longer fluidically communicating, but the drug reservoir is fluidically communicating. The user then can depress the plunger to administer the drug with confidence. Finally, the valve system can be turned such that both the drug reservoir and the dead-end pressure sensor are simultaneously in fluid communication with the vessel. In this scenario, the user may see a live read of the pressure as they depress the plunger. The drug may not enter the dead-end pressure sensor due to the lack of outlet in that direction.

Some embodiments incorporate a flow sensor, which, for example, can be used to provide real time volumetric flow rate data to the user such as during manual administration.

It should be noted that embodiments can be implemented in a wide variety of forms.

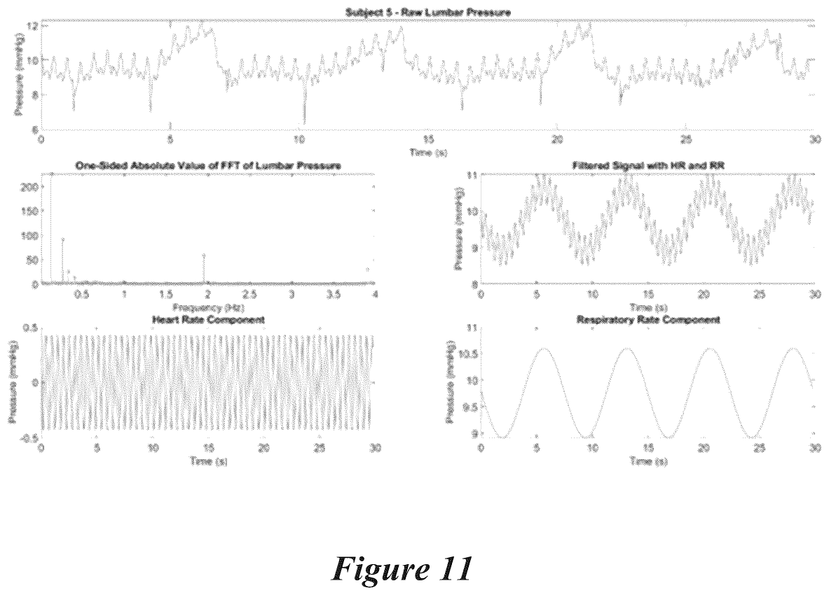

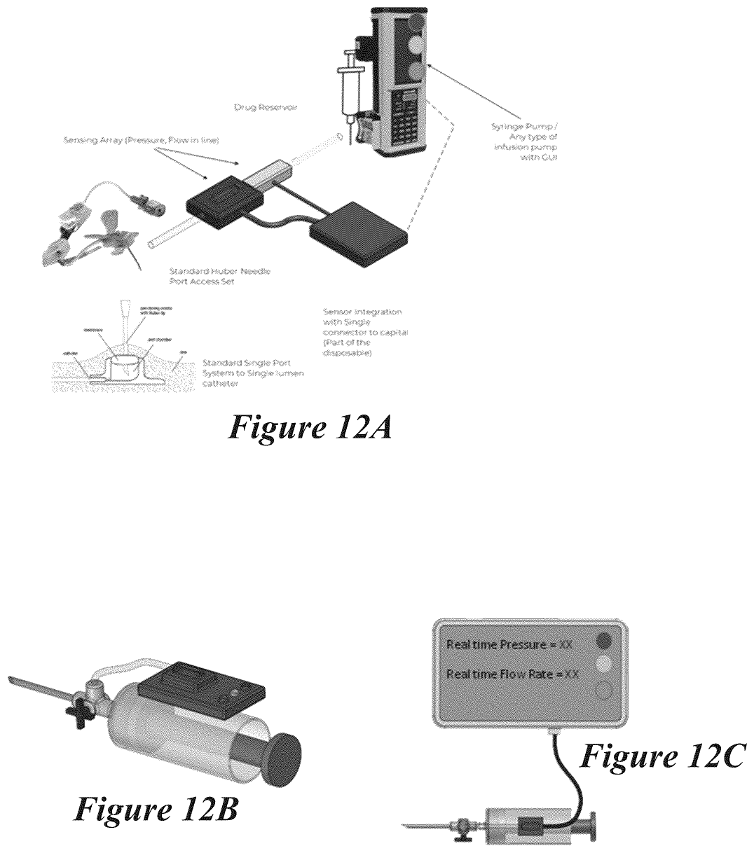

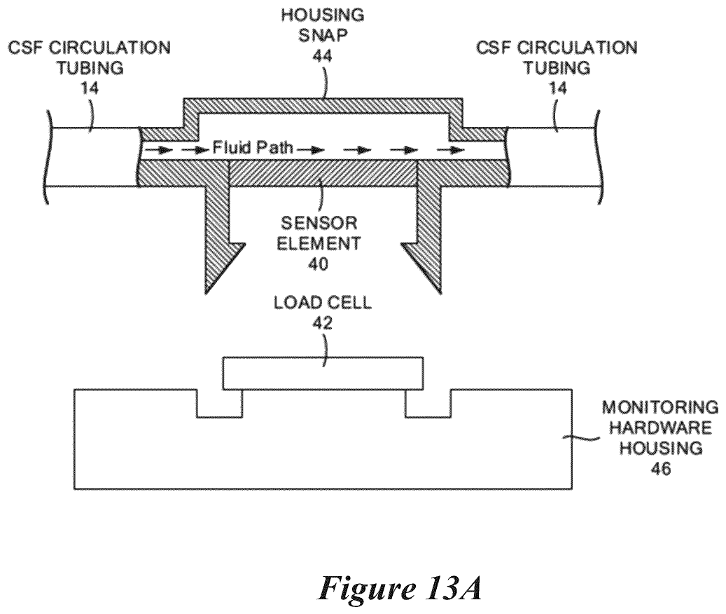

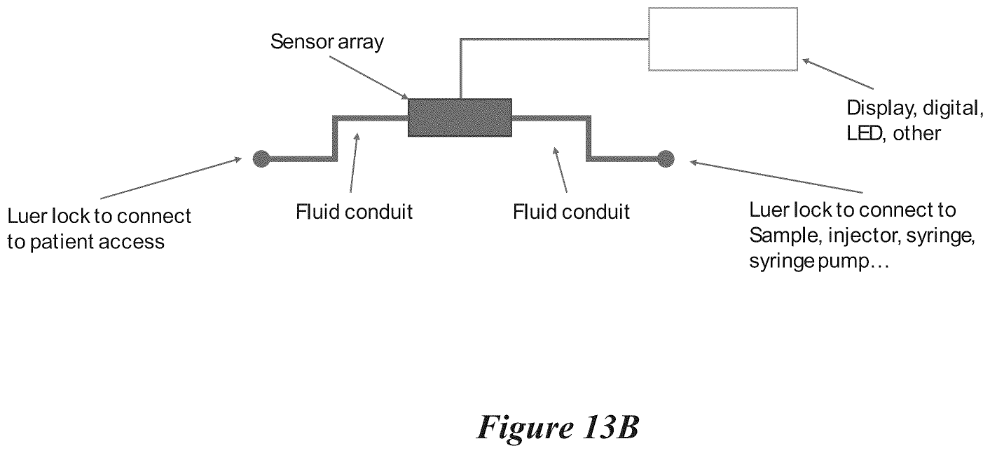

For one example, an embodiment can be implemented by the Enclear system in a distributed manner in which a remote sensor system conveys pressure waveform signals to the Enclear system for processing. The remote sensor system can be integrated into an intrathecal access device such as a catheter or syringe, an in-line fluid sensor such as depicted in FIG. 12A and in FIG. 13A), or other device having access to CSF with the ability to sense CSF pressure.

For another example, an embodiment can be implemented in a “smart” access device such as a catheter or syringe including a sensor that provides the pressure waveform signals, a processor that processes the pressure waveform signals to characterize a level of patency, and an output system to display an indication of the patency level (e.g., a green LED to indicate patency, a yellow LED to indicate inconclusive, and a red LED to indicate no patency, as depicted in FIG. 12B) or to convey the patency level information to a remote output device (e.g., to the Enclear system via a wired or wireless connection as depicted in FIG. 12C). The smart access device can be disposable and in some cases can include a needle such as would typically be used for lumbar punctures.

For another example, an embodiment can be implemented in a “smart” in-line fluid sensor including a sensor that provides the pressure waveform signals, a processor that processes the pressure waveform signals to characterize a level of patency, and an output system to display an indication of the patency level (e.g., a green LED to indicate patency, a yellow LED to indicate inconclusive, and a red LED to indicate no patency) or to convey the patency level information to a remote output device (e.g., to the Enclear system via a wired or wireless connection as depicted in FIG. 13B).

Embodiments can be implemented in other forms.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs or other programmable logic devices, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:
P1. A method comprising: receiving a heart rate signal of a patient; receiving a respiration rate signal of the patient; and using the heart rate signal and respiration rate signal to determine patency of an injection site.
P2. The method of innovation P1 wherein using comprises applying a Fast Fourier Transform to the heart rate signal and the respiration rate signal to produce two peaks, one peak being a low frequency peak and a second peak being a high frequency peak.
P3. The method of innovation P2 wherein using comprises applying a filter to the heart rate signal and the respiration rate signal.
P4. The method of innovation P3 wherein using further comprises counting the frequency of the filtered signals in real time after applying a filter.
P5. The method of innovation P3 wherein the filter is one or both of an infinite or finite response filter.
P6. The method of innovation P1 wherein using comprise fitting and approximating a sine wave to a time domain pressure signal as a sinusoidal regression, the regression having a correlation of determination (R2) above a certain minimum or threshold.
P7. The method of innovation P1 further comprising causing display of an interface displaying the determined patency.

The embodiments of the invention described above are intended to be merely exemplary; numerous variations and modifications will be apparent to those skilled in the art.

1. A method for determining a patency level for an intrathecal access site, the method comprising:
receiving a complex cerebrospinal fluid (CSF) pressure signal for an intrathecal access site;
processing the complex CSF pressure signal to extract a heart rate component and a respiratory rate component; and
processing the heart rate component and the respiratory rate component to determine a patency level for the intrathecal access site.

2. The method of claim 1, wherein the patency level is based on amplitude of at least one of the heart rate component or the respiratory rate component.

3. (canceled)

4. (canceled)

5. The method of claim 1, wherein processing the complex CSF pressure signal comprises passing the complex CSF pressure signal through a low-pass filter prior to processing.

6. The method of claim 1, wherein processing the complex CSF pressure signal comprises an FFT analysis to produce two peaks, one peak being a low frequency peak corresponding to the respiratory rate and a second peak being a high frequency peak corresponding to the heart rate, optionally including applying at least one of an infinite or finite response filter to the heart rate signal and the respiration rate signal and counting the frequency of the filtered signals in real time after applying the filter.

7. (canceled)

8. (canceled)

9. (canceled)

10. The method of claim 1, wherein processing the complex CSF pressure signal comprises a peak/valley process to derive the respiratory rate and the heart rate.

11. The method of claim 1, wherein processing the complex CSF pressure signal comprises fitting and approximating a sine wave to a time domain pressure signal as a sinusoidal regression, the regression having a correlation of determination (R2) above a predetermined minimum or threshold.

12. (canceled)

13. The method of claim 1, further comprising at least one of:
outputting an indication of the patency level, optionally wherein the indication includes at least one of a graphical indication (e.g., using words, color, bar graph, gauge, or other indication on a display device via a graphical user interface), a visual indication (e.g., turning on a light if patency if absent or lost, or using different color lights to indicate patency level such as green for patent, yellow for inconclusive, and red for no/inadequate patency), an audible indication (e.g., a buzzer if patency is absent/inadequate or lost, or a spoken audible indication of the patency level), or a tactile indication (e.g., a tactile alert such as vibration of a device worn by a caregiver if patency is absent or lost); or
automatically controlling a pump based on the patency level including at least one of automatically stopping the pump when patency level falls below a predetermined threshold or automatically controlling pump flow rate based on patency level (e.g. reduce flow rate if patency level is reduced or increase flow rate if patency level increases).

14. (canceled)

15. A computer program product comprising a tangible, non-transitory computer-readable medium having embodied therein computer program instructions which, when executed by at least one processor, perform computer processes for determining a patency level for an intrathecal access site, the computer processes comprising:
receiving a complex cerebrospinal fluid (CSF) pressure signal for an intrathecal access site;
processing the complex CSF pressure signal to extract a heart rate component and a respiratory rate component; and
processing the heart rate component and the respiratory rate component to determine a patency level for the intrathecal access site.

16. The computer program product of claim 15, wherein the patency level is based on amplitude of at least one of the heart rate component or the respiratory rate component.

17. (canceled)

18. (canceled)

19. The computer program product of claim 15, wherein processing the complex CSF pressure signal comprises passing the complex CSF pressure signal through a low-pass filter prior to processing.

20. The computer program product of claim 15, wherein processing the complex CSF pressure signal comprises an FFT analysis to produce two peaks, one peak being a low frequency peak corresponding to the respiratory rate and a second peak being a high frequency peak corresponding to the heart rate, optionally including applying at least one of an infinite or finite response filter to the heart rate signal and the respiration rate signal and counting the frequency of the filtered signals in real time after applying the filter.

21. (canceled)

22. (canceled)

23. (canceled)

24. The computer program product of 16 claim 15, wherein processing the complex CSF pressure signal comprises a peak/valley process to derive the respiratory rate and the heart rate.

25. The computer program product of 16 claim 15, wherein processing the complex CSF pressure signal comprises fitting and approximating a sine wave to a time domain pressure signal as a sinusoidal regression, the regression having a correlation of determination (R2) above a predetermined minimum or threshold.

26. (canceled)

27. The computer program product of claim 15, wherein the computer processes further comprise:
outputting an indication of the patency level, optionally wherein the indication includes at least one of a graphical indication (e.g., using words, color, bar graph, gauge, or other indication on a display device via a graphical user interface), a visual indication (e.g., turning on a light if patency if absent or lost, or using different color lights to indicate patency level such as green for patent, yellow for inconclusive, and red for no/inadequate patency), an audible indication (e.g., a buzzer if patency is absent/inadequate or lost, or a spoken audible indication of the patency level), or a tactile indication (e.g., a tactile alert such as vibration of a device worn by a caregiver if patency is absent or lost); or
automatically controlling a pump based on the patency level including at least one of automatically stopping the pump when patency level falls below a predetermined threshold or automatically controlling pump flow rate based on patency level (e.g. reduce flow rate if patency level is reduced or increase flow rate if patency level increases).

28. (canceled)

29. A device for determining a patency level for an intrathecal access site, the device comprising:
at least one processor configured to perform processes comprising:
receiving a complex cerebrospinal fluid (CSF) pressure signal for an intrathecal access site;
processing the complex CSF pressure signal to extract a heart rate component and a respiratory rate component; and
processing the heart rate component and the respiratory rate component to determine a patency level for the intrathecal access site.

30. The device of claim 29, wherein the patency level is based on amplitude of at least one of the heart rate component or the respiratory rate component.

31. The device of claim 29, wherein processing the complex CSF pressure signal comprises passing the complex CSF pressure signal through a low-pass filter prior to processing.

32. The device of claim 29, wherein processing the complex CSF pressure signal comprises an FFT analysis to produce two peaks, one peak being a low frequency peak corresponding to the respiratory rate and a second peak being a high frequency peak corresponding to the heart rate, optionally including applying at least one of an infinite or finite response filter to the heart rate signal and the respiration rate signal and counting the frequency of the filtered signals in real time after applying the filter.

33. (canceled)

34. (canceled)

35. (canceled)

36. The device of claim 29, wherein processing the complex CSF pressure signal comprises a peak/valley process to derive the respiratory rate and the heart rate.

37. The device of claim 29, wherein processing the complex CSF pressure signal comprises fitting and approximating a sine wave to a time domain pressure signal as a sinusoidal regression, the regression having a correlation of determination (R2) above a predetermined minimum or threshold.

38. (canceled)

39. (canceled)

40. (canceled)

41. (canceled)

42. (canceled)

43. The device of claim 29, further comprising a sensor system that provides the complex CSF pressure signal, optionally wherein the sensor system comprises an intrathecal access device or an in-line fluid sensor.

44. (canceled)

45. (canceled)

46. The device of claim 29, further comprising wherein the processes comprise least one of:
outputting an indication of the patency level, optionally wherein the indication includes at least one of a graphical indication (e.g., using words, color, bar graph, gauge, or other indication on a display device via a graphical user interface), a visual indication (e.g., turning on a light if patency if absent or lost, or using different color lights to indicate patency level such as green for patent, yellow for inconclusive, and red for no/inadequate patency), an audible indication (e.g., a buzzer if patency is absent/inadequate or lost, or a spoken audible indication of the patency level), or a tactile indication (e.g., a tactile alert such as vibration of a device worn by a caregiver if patency is absent or lost); or
automatically controlling a pump based on the patency level including at least one of automatically stopping the pump when patency level falls below a predetermined threshold or automatically controlling pump flow rate based on patency level (e.g. reduce flow rate if patency level is reduced or increase flow rate if patency level increases), wherein the device optionally includes the pump.

47. (canceled)

48. (canceled)

49. (canceled)

50. (canceled)

51. A system for determining a patency level for an intrathecal access site, the system comprising:
a sensor system that provides a complex cerebrospinal fluid (CSF) pressure signal for an intrathecal access site;
a processor system configured to process the complex CSF pressure signal to extract a heart rate component and a respiratory rate component and to process the heart rate component and the respiratory rate component to determine a patency level for the intrathecal access site; and
an output system configured to output an indication of the patency level.

52. The system of claim 51, wherein the patency level is based on amplitude of at least one of the heart rate component or the respiratory rate component.

53. The system of claim 51, wherein the sensor system comprises an intrathecal access device such as a catheter or syringe, optionally wherein the intrathecal access device further comprises a needle, or an in-line fluid sensor.

54. (canceled)

55. The system of claim 51, wherein processing the complex CSF pressure signal comprises passing the complex CSF pressure signal through a low-pass filter prior to processing.

56. The system of claim 51, wherein processing the complex CSF pressure signal comprises an FFT analysis to produce two peaks, one peak being a low frequency peak corresponding to the respiratory rate and a second peak being a high frequency peak corresponding to the heart rate, optionally including applying at least one of an infinite or finite response filter to the heart rate signal and the respiration rate signal and counting the frequency of the filtered signals in real time after applying the filter.

57. (canceled)

58. (canceled)

59. (canceled)

60. The system of claim 51, wherein processing the complex CSF pressure signal comprises a peak/valley process to derive the respiratory rate and the heart rate.

61. The system of claim 51, wherein processing the complex CSF pressure signal comprises fitting and approximating a sine wave to a time domain pressure signal as a sinusoidal regression, the regression having a correlation of determination (R2) above a predetermined minimum or threshold.

62. (canceled)

63. (canceled)

64. The system of claim 51, wherein the processor system and the output system are implemented in a first device and wherein the sensor system is implemented in a second device that is separate from the first device and that sends the complex CSF pressure signal to the first device over a wired or wireless communication system, optionally wherein the first device comprises at least one of a capital system or an infusion pump.

65. (canceled)

66. (canceled)

67. The system of claim 51, wherein the sensor system and the processing system are implemented in a first device and wherein the output system is implemented in a second device that is separate from the first device and that receives the indication of patency level from the first device, optionally wherein the first device comprises at least one of an intrathecal access device or an in-line fluid sensor and/or second device comprises at least one of a capital system, an infusion pump, or a display device.

68. (canceled)

69. (canceled)

70. (canceled)

71. (canceled)

72. (canceled)

73. The system of claim 51, wherein the sensor system, the processing system, and the output system are implemented in a single device, optionally wherein the single device comprises an intrathecal access device, an in-line fluid sensor, a capital device, or an infusion pump.

74. (canceled)

75. (canceled)

76. (canceled)

77. (canceled)

78. The system of claim 51, wherein the processor system is further configured to automatically control a pump based on the patency level including at least one of automatically stopping the pump when patency level falls below a predetermined threshold or automatically controlling pump flow rate based on patency level (e.g., reduce flow rate if patency level is reduced or increase flow rate if patency level increases), optionally wherein the system comprises the pump.

79. (canceled)
